(19) United States
(12) Patent Application Publication
(10) Pub. No.: US 2021/0155690 A1
(43) Pub. Date: May 27, 2021

(54) COMPOSITIONS AND METHODS FOR THE IMMUNE CHECKPOINT BLOCKADE OF TIM4

(71) Applicant: St. Jude Children's Research Hospital, Memphis, TN (US)

(72) Inventors: Douglas R. Green, Memphis, TN (US); Larissa Dias da Cunha, Memphis, TN (US)

(73) Assignee: St. Jude Children's Research Hospital, Memphis, TN (US)

(21) Appl. No.: 17/046,352

(22) PCT Filed: Apr. 9, 2019
(86) PCT No.: PCT/IB2019/052914

§ 371 Date: Oct. 9, 2020

Related U.S. Application Data
(60) Provisional application No. 62/656,587, filed on Apr. 12, 2018.

(51) Int. Cl. C07K 16/28; A61P 35/00; A61K 45/06; A61K 39/395

(52) U.S. Cl. CPC C07K 16/2803; A61P 35/00; C07K 2317/76; A61K 39/39541; C07K 2317/14; A61K 45/06

(57) ABSTRACT

Compositions and methods are provided for blocking Tim4 on the surface of myeloid cells in order to treat and prevent cancer in a subject. Blocking Tim4 on the surface of myeloid cells can restore anti-tumor immunity and reduce the uptake of apoptotic cells. Further, by administering an anti-Tim4 antibody, the pro-inflammatory anti-tumor response can be increased. Accordingly, methods for reducing the uptake of apoptotic cells and methods for increasing anti-tumor immunity are provided herein. The present disclosure further describes a method for producing monoclonal antibodies that bind Tim4 on the surface of myeloid cells. The anti-Tim4 antibodies can further be selected for their ability to inhibit engulfment of dead or dying cells, to elicit the production of inflammatory cytokines, and to restrict the growth of tumors.

### CROSS-REFERENCE TO RELATED APPLICATIONS

This application is a national stage filing under 35 U.S.C. 371 of PCT/IB2019/052914 filed Apr. 9, 2019, which International Application was published by the International Bureau in English on Oct. 17, 2019, and application claims priority from U.S. Provisional Patent Application No. 62/656,587, filed Apr. 12, 2018, which applications are hereby incorporated in their entirety by reference in this application.

### STATEMENT OF GOVERNMENT SUPPORT

This invention was made with government support under AI040646 awarded by the National Institutes of Health (NIH). The government has certain rights in the invention.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of S88435_0063_4_Seq_List.txt, a creation date of Oct. 8, 2020, and a size of 10.0 KB. The sequence listing filed via EFS-Web is part of the specification and is hereby incorporated in its entirety by reference herein.

### FIELD OF THE INVENTION

The invention relates to the field of cancer biology and immunology. In particular, the invention relates to a method for producing monoclonal antibodies that bind Tim4 on the surface of myeloid cells. The methods and compositions can be used to treat cancer and other cell proliferative disorders.

### BACKGROUND OF THE INVENTION

Cancer is the second leading cause of death in the United States, exceeded only by heart disease. Despite recent advances in cancer diagnosis and treatment, surgery and radiotherapy may be curative if a cancer is found early, but current drug therapies for metastatic disease are mostly palliative and seldom offer a long-term cure. Even with new chemotherapies entering the market, the need continues for new drugs effective in monotherapy or in combination with existing agents as first line therapy, and as second and third line therapies in treatment of resistant tumors.

Cancer cells are by definition heterogeneous. For example, within a single tissue or cell type, multiple mutational “mechanisms” may lead to the development of cancer. As such, heterogeneity frequently exists between cancer cells taken from tumors of the same tissue and same type that have originated in different individuals. Frequently observed mutational “mechanisms” associated with some cancers may differ between one tissue type and another (e.g., frequently observed mutational “mechanisms” leading to colon cancer may differ from frequently observed “mechanisms” leading to leukemias). It is therefore often difficult to predict whether a particular cancer will respond to a particular chemotherapeutic agent.

Surrounding a nest of neoplastic tumor cells is a microenvironment consisting of diverse mesenchymal support cells (fibroblasts, adipocytes, etc.), cells forming hematogenous and lymphatic vasculature, immune cells, and a dynamically-regulated extracellular matrix (ECM), all of which influence neoplastic progression to the malignant and/or metastatic state. Resident or recruited immune cells present within the tumor microenvironment (TME) represent a diverse assemblage of both lymphoid and myeloid cells, and depending on their activation state and phenotype, can either promote or inhibit various aspects of tumor development as well as regulate response to anti-cancer therapy

Myeloid cells, including various subsets of monocytes, neutrophils, and macrophages, are implicated in T cell suppression. Macrophages exist along a continuum of subtypes in tumors with classically-activated M1-like macrophages on one end, and alternatively-activated M2-like macrophages on the other. Macrophages present in several types of solid tumors are predominantly immunosuppressive (M2-like) and play a major role in suppressing the actions of CD8⁺ T cells, as well as fostering malignancy by providing pro-growth, survival, and angiogenic molecules critical for rapid tumor development

In healthy subjects, immune checkpoint pathways (also known as immune-inhibitory pathways) are important for maintaining self-tolerance and preventing autoimmunity. However, immune checkpoint pathways in cancer cells are often dysregulated, leading to the ability of tumors to evade the body's endogenous anti-tumor immune response. Cancers co-opt the immune checkpoint pathways in a number of ways, such as upregulating the expression of immune checkpoint proteins that normally serve immune-inhibitory roles. For example, inhibitory ligands and receptors that regulate T cell effector activity are often overexpressed in cancer cells.

The blockade of immune checkpoints in cancer immunotherapy has emerged as a promising approach to combat this mechanism by which cancer cells evade the anti-tumor immune response. For example, antibodies directed against immune-inhibitory proteins, such as immune checkpoint proteins are being explored as potential anti-cancer therapeutics. See, Pardoll. Nat. Reviews Cancer. (2012) 12:252-264.

Existing immune checkpoint blockades for cancer treatment target molecules that are either on T cells or that interact with molecules on T cells. In contrast, Tim4 is on myeloid cells and functions in the uptake and clearance of dying cells. Thus, targeting Tim4 presents a novel approach to enhancing anti-tumor immunity and treating cancer.

### SUMMARY OF THE INVENTION

Compositions and methods are provided for blocking Tim4 on the surface of myeloid cells in order to treat and prevent cancer in a subject. Blocking Tim4 on the surface of myeloid cells can restore anti-tumor immunity and reduce the uptake of apoptotic cells. Further, by administering an anti-Tim4 antibody, the pro-inflammatory anti-tumor response can be increased. Accordingly, methods for reducing the uptake of apoptotic cells and methods for increasing anti-tumor immunity are provided herein.

The present disclosure further describes a method for producing monoclonal antibodies that bind Tim4 on the surface of myeloid cells. Monoclonal antibodies that bind recombinant human or murine Tim4 can be tested and selected for their ability to bind recombinant Tim4. Those antibodies that bind recombinant Tim4 can then be selected for their ability to inhibit or block the uptake of apoptotic cells by macrophages expressing endogenous or recombinant Tim4. Those antibodies that can block the engulfment of apoptotic cells can increase the pro-inflammatory response. Finally, antibodies can be tested for the ability to resutrct the growth of tumors.

### BRIEF DESCRIPTION OF THE FIGURES

FIG. 1 depicts the quantification of micrometastatic spots on the lungs of Tim4^{-/-} and littermate Tim4^{+/+} mice. Spots were counted 21 days after intravenous injection of 10⁵ B16F10 cells. Dots represent each individual mouse, line indicates mean and error bars represent s.e.m. and significance was calculated using Student's t-test with Welch's correction. Data are representative of two independent experiments, *p<0.05, **p<0.01, ***p<0.001.

FIG. 2 shows the tumor growth in wild-type and deficient littermates injected with 10⁵ LLC cells subcutaneously (Tim4^{+/+} n=6, Tim4^{-/-} n=8). Data are representative of two independent experiments, *p<0.05, **p<0.01, ***p<0.001.

FIG. 3 also shows the tumor growth in wild-type and deficient littermates injected with 10⁵ LLC cells subcutaneously (Atg7^{f/f} Cre⁻ n=7, Atg7^{f/f} Cre⁺ n=4/Atg7^{f/f} Ifnar^{-/-} Cre⁻ n=10, Atg7^{f/f} Ifnar^{-/-} Cre⁺ n=10). Data are representative of two independent experiments, *p<0.05, **p<0.01, ***p<0.001.

FIG. 4 shows the production of IFNg by CD8+ lymphocytes isolated from subcutaneous LLC tumors.

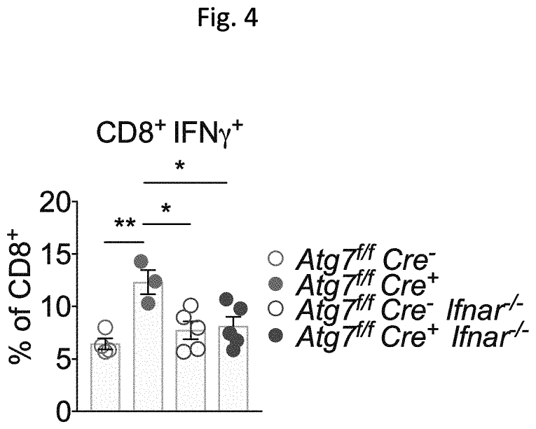

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Compositions and methods are provided herein for the neutralization of Tim4 on the surface of myeloid cells. Tim4 belongs to the T-cell immunoglobulin and mucin domain (Tim) gene family, which participates in immunoregulation. As used herein, “Tim4”, “Tim 4”, and “Tim-4” refer to a protein of SEQ ID NO: 2 (human) or SEQ ID NO: 4 (mouse), or an active fragment or variant thereof, and encoded by a nucleic acid molecule having SEQ ID NO: 1 (human) or SEQ ID NO: 3 (mouse), or an active fragment or variant thereof. In some embodiments, “Tim4” is known as hepatitis A virus cellular receptor 1. Tim4 can be found naturally on the surface of myeloid cells and may participate in the engulfment of dead and dying cells. The compositions and methods disclosed herein take advantage of Tim4 as a novel immune checkpoint to neutralize and block Tim4 in an effort to enhance T cell immune response to cancer.

As used herein, “immune checkpoints” refer to any molecule of the immune system that can enhance or depress an immune signal. Immune checkpoints that protect against excessive activation of T cells (CTLA-4) and deleterious T cell effector function (such as PD-1/PD-L1) are hijacked during malignant tumor growth to inhibit effective immune response against tumor. Innate immune cells also participate in the regulation of the immune responses against tumor cells. Sensing of signals from dying tumor cells, including engulfment of cell corpses, is a known mechanism that induces anti-inflammatory function of myeloid cells, promoting tumor tolerance, and thereby providing another mechanism of immune checkpoint that can be manipulated by cancer cells. Myeloid cells can inhibit priming and effector function of tumor-reactive T cells. Thus, by blocking the manipulation of immune checkpoints by cancer cells using a molecule such as a specific antibody, proper immune function can be restored and the T cell response to the cancer cell can be increased. Proper T cell response to cancer cells can increase the inflammatory response and the direct elimination of tumor cells by CD8⁺ cytolytic cells. Further, by blocking Tim4 on the surface of myeloid cells, the engulfment of apoptotic cells or dying cells by macrophages can be inhibited.

### II. Pharmaceutical Compositions Comprising Anti-Tim4 Antibodies

Provided herein are compositions comprising an effective amount of an anti-Tim4 antibody. In some embodiments the anti-Tim4 antibody is produced or selected using the methods disclosed elsewhere herein. An “anti-Tim4” or “anti-Tim-4” antibody, as used herein, refers to an antibody that specifically or preferentially binds Tim4 or peptides of Tim4 (i.e., Tim4 peptides) on the surface of myeloid cells. Tim4 peptides bound by an anti-Tim4 antibody can be any fragment or variant of Tim4. In specific embodiments, Tim4 peptides include a protein domain or binding domain conserved within Tim4. Tim4 peptides can be at least 10, 15, 20, 25, 30, 35, 40, 45, 50, 60, 70, 80, 90, 100, 150, 200, 250, or up to the full length of the naturally occurring Tim4 protein. In specific embodiments, Tim4 peptides are about 200-600, about 300-500, about 350-450, or about 360-400 amino acids in length. In specific embodiments, Tim4 peptides retain Tim4 activity. As used herein, Tim4 activity refers to the ability of Tim4 or a Tim4 peptide on a myeloid cell to alter the activity or expression of the associated myeloid cell.

As used herein, the term “antibody” refers to a polypeptide or a polypeptide complex that specifically recognizes and binds to an antigen (e.g., Tim4) through one or more immunoglobulin variable regions. An antibody can be a whole antibody, an antigen binding fragment or a single chain thereof. The terms “antibody fragment” or “antigen-binding fragment” are used with reference to a portion of an antibody, such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered proteins comprising immunoglobulin variable regions that act like an antibody by binding to a specific antigen to form a complex.

A “single-chain fragment variable” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. As used herein, the terms “VH1” and “VH2” refer to immunoglobulin heavy chain variable domains corresponding to two different binding specificities. Likewise, the terms “VL1” and “VL2” refer to light chain variable domains corresponding to two different binding specificities. When used together, it is to be understood that VH1 and VL1 regions define a common binding specificity and that VH2 and VL2 domains define a second binding specificity.

The term “antibody” encompasses various broad classes of polypeptides that can be distinguished biochemically. Those skilled in the art will appreciate that heavy chains are classified as alpha, delta, epsilon, gamma, and mu, or α, δ, ε, γ, and μ, with some subclasses among them (e.g., .gamma.1-.gamma.4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are within the scope of the present disclosure.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′, F(ab′)₂, Fd, Fvs, single-chain Fvs (scFv), single-chain antibodies, disulfide-linked Fvs (sdFv), fragments comprising either a VL or VH domain, fragments produced by a Fab expression library and anti-idiotypic (anti-Id) antibodies. Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains in conventional antibodies increases as they become more distal from the antigen-binding site or amino-terminus of the antibody. In conventional antibodies, the N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens (e.g., Tim4). That is, the VL domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VL chains (i.e. CDR-H1, CDR-H2, CDR-H3, CDR-L1, CDR-L2 and CDR-L3). In some instances, e.g., certain immunoglobulin molecules are derived from camelid species or engineered based on camelid immunoglobulins. Alternatively, an immunoglobulin molecule may consist of heavy chains only, with no light chains or light chains only, with no heavy chains.

Antibodies disclosed herein may be from any animal origin, including birds and mammals. Preferably, the antibodies are human, murine, donkey, rabbit, goat, guinea pig, camel, llama, horse, or chicken antibodies. In some embodiments, the variable region may be condricthoid in origin (e.g., from sharks). As used herein, the phrase “humanized antibody” refers to an antibody derived from a non-human antibody, typically a mouse monoclonal antibody. Alternatively, a humanized antibody may be derived from a chimeric antibody that retains or substantially retains the antigen binding properties of the parental, non-human, antibody but which exhibits diminished immunogenicity as compared to the parental antibody when administered to humans. As used herein, the phrase “chimeric antibody,” refers to an antibody where the immunoreactive region or site is obtained or derived from a first species and the constant region (which may be intact, partial or modified in accordance with the instant disclosure) is obtained from a second species. In certain embodiments the target binding region or site will be from a non-human source (e.g., mouse or primate) and the constant region is human.

In specific embodiments, the antibodies disclosed herein are specifically or preferentially bind Tim4 or Tim4 peptides. By “specifically binds” or “has specificity to”, it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.” As used herein, an antibody that Preferentially binds an antigen (e.g., Tim4 or Tim4 peptides) primarily binds the antigen, but does not necessarily solely bind the antigen. An antibody that preferentially binds an antigen has less specificity for the antigen than an antibody that specifically binds the same antigen.

The antibodies disclosed herein function to block or neutralize the immune checkpoint regulator Tim4 on the surface of myeloid cells. As used herein, the phrase “immune checkpoint regulator” refers to a functional class of agents, which inhibit or stimulate signaling through an immune checkpoint regulator. An “immune checkpoint regulator” includes receptors and their associated ligands, which together provide a means for inhibiting or stimulating signaling pathways that otherwise lead to T-cell activation or cell engulfment. Blocking Tim4 on the surface of myeloid cells can prevent the binding of immune checkpoint binding agonists and prevent suppression of the immune response to tumor cells. Accordingly, the term “block” or “blocking” refers to the binding of Tim4 by an antibody or antagonist disclosed herein. Blocking can refer to partial or complete binding of an antibody or other antagonist to Tim4. Blocking of Tim4 can prevent reduction of T cell or myeloid cell signaling by cancer cells or molecules associated with cancer cells. In some embodiments, signaling of T cells or myeloid cells can be stimulated by the blocking or binding of Tim4 by an antibody or other antagonist disclosed herein, thereby increasing anti-tumor immunity.

The phrases “immune checkpoint binding antagonist” and “immune checkpoint antagonist” are used interchangeably herein with reference to a class of immune checkpoint regulators that interfere with (or inhibit) the activity of an immune checkpoint regulator so that, as a result of the binding to the checkpoint regulator or its ligand, signaling through the checkpoint regulator receptor is blocked or inhibited. By inhibiting this signaling, immune-suppression can be reversed so that T cell immunity against cancer cells can be re-established or enhanced or proper engulfment activity by myeloid cells can be restored or enhanced. Immune checkpoint antagonists can include antibody fragments, peptide inhibitors, dominant negative peptides and small molecule drugs, either in isolated forms or as part of a fusion protein or conjugate. In specific embodiments, an immune checkpoint antagonist is an antibody that binds Tim4 on the surface of myeloid cells.

Although immune checkpoint molecules are typically associated with T cells, the immune checkpoint molecule Tim4 is found on myeloid cells. As used herein, myeloid cells refer to granulocytes and monocytes that differentiate from common progenitors in the bone marrow. Examples of myeloid cells include monocytes, macrophages, dendritic cells, neutrophils, eosinophils, and basophils. The major function of the myeloid lineage cells (e.g., neutrophils and macrophages) is the phagocytosis of infectious organisms, live unwanted damaged cells, senescent and dead cells (apoptotic and necrotic), as well as the clearing of cellular debris. In specific embodiments, blocking of Tim4 on the surface of myeloid cells can reduce the engulfment or phagocytosis of apoptotic cells by the myeloid cells. Reduction of engulfment of apoptotic cells can promote and increase anti-tumor immunity and induce immunogenic cell death. Reduction or inhibition of engulfment or phagocytosis refers to a statistically significant reduction of uptake, engulfment, or phagocytosis. Such decreases or reductions can include, for example, at least a 5%, 10%, 20%, 30%, 40%, 50%, 60%, 70%, 80%, 90%, 95%, 100% or more decrease in the measured or observed level of uptake, engulfment or phagocytosis.

As used herein, “engulfment” or “phagocytosis” of cells refers to the engulfment and digestion of such cells by myeloid cells, such as macrophages, and the eventual digestion or degradation of these cells and their release extracellularly, or intracellularly, to undergo further processing. Engulfment and phagocytosis can be measured by flow cytometry, immunofluorescence microscopy methodologies, or any other method known in the art.

Apoptosis is an ordered and orchestrated cellular process that occurs in physiological and pathological conditions. As used herein, apoptotic cells are cells that have started the process of apoptosis. Characterization of apoptotic cell death can be performed by staining of cells with Annexin V and/or Propidium Iodide staining. In some embodiments, the apoptotic cells are thymocytes, cancerous thymocytes, or a tumor-derived cell line. Any tumor-derived cell line can be an apoptotic cell, including cell lines derived from melanoma, thymoma, lewis lung carcinoma, lymphoma and adenocarcinomas. Accordingly, in specific embodiments, blocking Tim4 on the surface of myeloid cells can reduce the uptake and engulfment of apoptotic cells, and particularly reduce the engulfment of apoptotic thymocytes or tumor derived cell lines. In particular embodiments, blocking Tim4 on the surface of myeloid cells can reduce the uptake and/or engulfment of apoptotic cells in vivo, in vitro, and/or ex vivo.

The methods and compositions disclosed herein can be used to block Tim4 on the surface of myeloid cells in order to increase or restore anti-tumor immunity. In specific embodiments, anti-tumor immunity can be increased or restored by preventing suppression by cancer cells of a subject's anti-tumor immune response. The term “antitumor immunity” or “anti-tumor immunity” broadly refers to innate and adaptive immune responses which lead to tumor control. Particular aspects anti-tumor immunity include the processing and presentation of released antigens by antigen-presenting cells (APCs), interaction with T lymphocytes, subsequent immune/T-cell activation, trafficking of antigen-specific effector cells, and the engagement of the target tumor cell by the activated effector T cell. Anti-tumor immunity can be imposed by antigen-specific CD8⁺ T cells and tumoricidal macrophages. Antigens (Ag), typically foreign substances of environmental, viral, or bacterial origin, products of somatically altered proteins, or debris from dying (apoptotic) cells are processed and presented by major histocompatibility complex (MHC) on antigen presenting cells, including (but not limited to) dendritic cells, macrophages, and B cells. CD8⁺ T cells utilize T cell receptors (TCRs) to recognize WIC-presented peptides and subsequently mount an antigen-specific cytolytic attack. In particular, Ag-TCR engagement ultimately leads to activation and proliferation of CD8⁺ T cells that play a crucial role in autoimmunity, response to pathogens, and tumor suppression. Also, myeloid cells, including various subsets of monocytes, neutrophils, and macrophages, are implicated in T cell suppression. Anti-tumor immunity can be measured, in some embodiments, by measuring the effect of administration of the anti-Tim4 antibody or antibody fragment disclosed herein on tumor size, rate of tumor growth, rate of tumor appearance, or tumor phenotype.

In specific embodiments, increased anti-tumor immunity can be identified by measuring at least one of: increased CD8+ T cells, increased CD4+ T cells, increased CD4+ or CD8+ T cells secreting interferon gamma, and/or decreased CD4+ T cells secreting IL10 among tumor-infiltrating lymphocytes. As used herein an increase in CD8+ T cells, CD4+ T cells, CD4+ or CD8+ T cells secreting interferon gamma refers to a statistically significant increase. Such an increase can include, for example, at least a 5%, 10%, 20%, 30%, 40%, 50%, 60%, 70%, 80%, 90%, 95%, 100% or more increase in the measured or observed level of CD8+ T cells, CD4+ T cells, CD4+ or CD8+ T cells secreting interferon gamma. As used herein, a decrease in CD4+ T cells secreting IL10 among tumor-infiltrating lymphocytes refers to a significant reduction of CD4+ T cells secreting IL10 among tumor-infiltrating lymphocytes. Such decreases or reductions can include, for example, at least a 5%, 10%, 20%, 30%, 40%, 50%, 60%, 70%, 80%, 90%, 95%, 100% or more decrease in the measured or observed level of CD4+ T cells secreting IL10 among tumor-infiltrating lymphocytes.

In specific embodiments, anti-tumor immunity can be measured by identifying an increase in the level of pro-inflammatory cytokines. Specifically, administration of an antibody to block Tim4 on the surface of myeloid cells can increase anti-tumor immunity by increasing pro-inflammatory cytokine production. As used herein, an increase in the level of pro-inflammatory cytokine production comprises any statistically significant increase in the level of pro-inflammatory cytokine production in a subject when compared to an appropriate control. Such increases can include, for example, at least a 5%, 10%, 20%, 30%, 40%, 50%, 60%, 70%, 80%, 90%, 100% or more increase in the level of proinflammatory cytokines. Proinflammatory cytokines include, but are not limited to IL1-alpha, IL1-beta (IL-1β), TNF-alpha (TNF-α), IL-2, IL-3, IL-6, IL-7, IL-9, IL-12, IL-17, IL-18, TNF-alpha, LT, LIF, Oncostatin, or IFN-alpha, IFN-beta, IFN-gamma. Methods to assay for cytokine levels are known and include, for example Leng S., et al. (2008) J Gerontol A. Biol Sci Med Sci 63(8): 879-884. Methods to assay for the production of pro-inflammatory cytokines include multiplex bead assay, ELISPOT and flow cytometry. See, for example, Maecker et al. (2005) BMC Immunology 6:13.

Inflammatory cytokine production can also be measured by assaying the ratio of pro-inflammatory cytokine production to anti-inflammatory cytokine production. In specific aspects, the ratio of pro-inflammatory cytokine production to anti-inflammatory cytokine production is increased by about 1, 2, 3, 4, 5, 6, 7, 8, 9, 10, 20, 30, 40, 50, 60, 70, 80, 90, 100, 300, 600, 900, 1000 fold or greater when compared to an appropriate control. In other aspects, the ratio of pro-inflammatory cytokine production to anti-inflammatory cytokine production is increased by about 1 to 5 fold, about 5 to 10 fold, about 10 to 20 fold, about 20 to 30 fold, about 30 to 40 fold, about 40 fold to 60 fold, about 60 fold to 80 fold, about 80 fold to about 100 fold, about 100 to 200 fold, about 200 fold to 300 fold, about 300 to 400 fold, about 400 to about 500 fold, about 500 to about 500 fold, about 500 fold to about 700 fold, about 700 fold to 800 fold, about 800 fold to about 1000 fold or greater when compared to an appropriate control. Methods to determine the ratio of pro-inflammatory cytokine production to anti-inflammatory cytokine production can be found, for example, Leng S., et al. (2008) J Gerontol, A. Biol Sci Med Sci 63(8): 879-884. Methods to assay for the production of cytokines include multiplex bead assay, ELISPOT and flow cytometry. See, for example, Maecker et al. (2005) BMC Immunology 6:13.

### 2. Methods of Treatment

Provided herein are methods of decreasing or treating cancer in a subject having cancer. “Treatment” or “treating” as used herein refers to curing, healing, alleviating, relieving, altering, remedying, ameliorating, improving, or affecting the condition or the symptoms of a cancer or cell proliferative disorder in a subject by blocking Tim4 or a Tim4 peptide on the surface of a myeloid cell. As used herein the term “symptom” refers to an indication of disease, illness, injury, or that something is not right in the body.

Symptoms are felt or noticed by the individual experiencing the symptom, but may not easily be noticed by others. Others are defined as non-health-care professionals. Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body. As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

Sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage. Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma. Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease. Along with cancers of the skin, some internal cancers can cause skin signs that can be seen. These changes include the skin looking darker (hyperpigmentation), yellow (jaundice), or red (erythema); itching; or excessive hair growth. In some cases, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could be a symptom of cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer. Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size. Indigestion or trouble swallowing could be symptomatic of cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma. A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant invention.

In specific embodiments, decreasing cancer, treatment or treating cancer encompasses a reduction in the size of a tumor disclosed herein. Tumor size can be determined using a variety of methods known in the art, such as, for example, by measuring the dimensions of tumor(s) upon removal from the subject, e.g., using calipers, or while in the body using imaging techniques, e.g., ultrasound, computed tomography (CT) or magnetic resonance imaging (MRI) scans. Tumor size can be determined, for example, by determining tumor weight or tumor volume. As used herein, a reduction of tumor size refers to a rejection of the tumor diameter or tumor volume. The decrease in size can be, for example, a decrease of tumor diameter of 0.01 mm, 0.05 mm, 0.10 mm, 0.12 mm, 0.14 mm, 0.16 mm, 0.18 mm, 0.20 mm, 0.25 mm, 0.30 mm, 0.35 mm, 0.40 mm, 0.45 mm, 0.50 mm, 0.6 mm, 0.7 mm, 0.8 mm, 0.9 mm, 1.0 mm, 1.1 mm, 1.2 mm, 1.3 mm, 1.4 mm, 1.5 mm, 1.75 mm, 2.0 mm, 3.0 mm, 4.0 mm, 5.0 mm, 6.0 mm, 7.0 mm, 8.0 mm, 9.0 mm, 10.0 mm or more. The decrease in size can be a decrease in tumor volume of 10 mm³, 20 mm³, 30 mm³, 40 mm³, 50 mm³, 75 mm³, 100 mm³, 150 mm³, 200 mm³, 250 mm³, 300 mm³, 350 mm³, 400 mm³, 500 mm³, 600 mm³, 700 mm³, 800 mm³, 900 mm³, 1000 mm³ or more. In specific embodiments, such decreases or reductions in tumor size can be, for example, at least a 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% reduction in tumor size. In specific embodiments, treatment or treating encompasses a reduction in the number of tumors in a subject. The decrease in tumor number can be a decrease of at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or more tumors in a subject.

In some embodiments, treatment or treating encompasses a reduction in the spread or the progression of a cancer. The spread or progression of cancer can be reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a proper control. The spread or progression of cancer can be determined by measuring the tumor size, tumor number, tumor location, or any other method known in the art for measuring spread or progression of cancer.

Treating cancer can result in a decrease in number of metastatic lesions in other tissues or organs distant from the primary tumor site. Preferably, after treatment, the number of metastatic lesions is reduced by 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% compared to the number of metastatic lesions prior to administration of a anti-Tim4 antibody. The number of metastatic lesions may be measured by any reproducible means of measurement. The number of metastatic lesions may be measured by counting metastatic lesions visible to the naked eye or at a specified magnification.

Treating cancer can result in an increase in average survival time of a population of treated subjects in comparison to a population of untreated subjects. In some embodiments, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a anti-Tim4 antibody. Preferably, the mortality rate is decreased by more than 2%, 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. A decrease in the mortality rate of a population of treated subjects may be measured by any reproducible means. A decrease in the mortality rate of a population may be measured, for example, by calculating for a population the average number of disease-related deaths per unit time following initiation of treatment with an anti-Tim4 antibody. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% relative to the rate prior to administration of the anti-Tim4 antibody. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in the number or proportion of cells having an abnormal appearance or morphology. Preferably, after treatment, the number of cells having an abnormal morphology is reduced by at least 5% %, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% relative to the same measurement prior to treatment with an anti-Tim4 antibody. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleomorphism.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As described herein, a composition that is specific for Tim4 on the surface of myeloid cells can be administered in an effective amount in order to treat the cancer or cell proliferative disorder in the subject. In specific embodiments, the composition that is specific for Tim4 is an anti-Tim4 antibody. In certain embodiments, an “effective amount” or a “therapeutically effective amount” of an anti-Tim4 antibody can be sufficient to achieve a desired clinical result, including but not limited to, for example, ameliorating disease, stabilizing a subject, preventing or delaying the development of, or progression of, a proliferative disease, disorder, or condition in a subject. In specific embodiments, an effective amount is any amount sufficient to treat cancer or a cell proliferative disorder as described herein. For example, an effective amount is any amount of an anti-Tim4 antibody sufficient to reduce the tumor size, tumor number, reduce tumor spread, or reduce the progression of a cancer or cell-proliferative disorder. An effective amount of therapy can be determined based on one administration or repeated administration. Methods of detection and measurement of the indicators above are known to those of skill in the art. Such methods include, but are not limited to measuring reduction in tumor burden, reduction of tumor size, reduction of tumor volume, reduction in proliferation of secondary tumors, decreased solid tumor vascularization, expression of genes in tumor tissue, presence of biomarkers, lymph node involvement, histologic grade, and nuclear grade. “Positive therapeutic response” refers to, for example, improving the condition of at least one of the symptoms of a cancer, decreasing tumor size or tumor number, and/or reducing the progression of the cancer or cell proliferation disorder.

The specific therapeutically effective dose level for any particular subject will depend upon a variety of factors including the disorder being treated and the severity of the disorder; activity of the specific anti-Tim4 antibody employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al., (2004), Applied Therapeutics: The Clinical Use of Drugs, Lippincott Williams & Wilkins, ISBN 0781748453; Winter, (2003), Basic Clinical Pharmacokinetics, 4.sup.th ed., Lippincott Williams & Wilkins, ISBN 0781741475; Sharqel, (2004), Applied Biopharmaceutics & Pharmacokinetics, McGraw-Hill/Appleton & Lange, ISBN 0071375503). For example, it is well within the skill of the art to start doses of agents at levels lower than those required to achieve the desired therapeutic effect and to gradually increase the dosage until the desired effect can be achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of compositions described herein can occur as a single event, a periodic event, or over a time course of treatment. For example, agents can be administered daily, weekly, bi-weekly, or monthly. As another example, agents can be administered in multiple treatment sessions, such as 2 weeks on, 2 weeks off, and then repeated twice; or every 3rd day for 3 weeks. A first composition including a ligand coupled to molecule or substrate and a second composition including a receptor coupled to a radioisotope can have the same or different administration protocols. One of ordinary skill will understand these regimes to be exemplary and could design other suitable periodic regimes. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

A. Treatment of Cancer

Methods and compositions are provided herein for decreasing or treating cancer in a subject having cancer. As used herein, “cancer” refers to any cell-proliferative disorder in which unregulated or abnormal growth, or both, of cells can lead to the development of an unwanted condition or disease. Exemplary cell proliferative disorders of the invention encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. A cell proliferative disorder includes a non-cancer condition or disorder in which the division and progression of cells is uncontrolled. Preferably, the methods provided herein are used to treat or alleviate a symptom of cancer. The term “cancer” includes solid tumors, as well as, hematologic tumors, and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

As used herein, a “normal cell” is a cell that cannot be classified as part of a “cell proliferative disorder”, “cancer”, or “tumor”. A normal cell lacks unregulated or abnormal growth, or both, that can lead to the development of an unwanted condition or disease. Preferably, a normal cell possesses normally functioning cell cycle checkpoint control mechanisms.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; skin-related hyperproliferative disorders, psoriasis; eczema; atopic dermatitis; hyperpigmentation disorders, eye-related hyperproliferative disorders, age-related macular degeneration, ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; fibroadipose hyperplasia; spinocerebullar ataxia type 1; CLOVES syndrome; Harlequin ichthyosis; macrodactyly syndrome; Proteus syndrome (Wiedemann syndrome); LEOPARD syndrome; systemic sclerosis; Multiple Sclerosis; lupus; fibromyalgia; AIDS and other viral diseases such as Herpes Zoster, Herpes Simplex I or II, influenza virus and cytomegalovirus; diabetes mellitus; hemihyperplasia-multiple lipomatosis syndrome; megalocephaly; rare hypoglycemia, Klippel-Trenaunay syndrome; harmatoma; Cowden syndrome; or overgrowth-hyperglycemia.

Exemplary cancers include, but are not limited to, adrenocortical carcinoma, AIDS-related cancers, AIDS-related lymphoma, anal cancer, anorectal cancer, cancer of the anal canal, anal squamous cell carcinoma, angiosarcoma, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, head and neck squamous cell carcinoma, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, T-cell lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, lung squamous cell carcinoma, AIDS-related lymphoma, non-Hodgkin lymphoma, primary central nervous system lymphoma, B-cell lymphoma, primary effusion lymphoma, Waldenstram macroglobulinemia, medulloblastoma, melanoma, intraocular (eye) melanoma, merkel cell carcinoma, lewis cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, pancreatic endocrine tumor, paranasal sinus and nasal cavity cancer, parathyroid cancer, cholangiocarcinoma, penile cancer, pharyngeal cancer, pheochromocytoma, pineoblastoma and supratentorial primitive neuroectodermal tumors, pituitary tumor, pituitary adenoma, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

In particular embodiments, the cancer is attacked by T cell mediated immunity. In specific embodiments, the cancer is lymphoma, melanoma, colon carcinoma, mammary carcinoma, lung carcinoma, fibrosarcoma, renal carcinoma, neuroblastoma, or ovarian carcinoma. For example, the cancer can be a lymphoma tumor, melanoma tumor, colon carcinoma tumor, mammary carcinoma tumor, lung carcinoma tumor, fibrosarcoma tumor, renal carcinoma tumor, neuroblastoma tumor, ovarian carcinoma tumor, or Lewis cell carcinoma (Lewis lung carcinoma). In specific embodiments, the canner is a melanoma, such as a B16F10 melanoma, or a Lewis Lung Carcinoma.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

As used herein, a “subject” or “subject in need thereof” is a subject having a cell proliferative disorder, or cancer or a subject having an increased risk of developing a cell proliferative disorder or cancer relative to the population at large. A subject in need thereof can have a precancerous condition. In specific embodiments, a subject in need thereof has cancer. A “subject” includes a mammal. The mammal can be e.g., any mammal, e.g., a human, primate, bird, mouse, rat, fowl, dog, cat, cow, horse, goat, rabbit, camel, sheep or a pig. In certain embodiments, the mammal is a human. In some embodiments, the human undergoing treatment can be a newborn, infant, toddler, preadolescent, adolescent, or adult.

In particular embodiments, the subject has a cancer that interacts with Tim4 on the surface of myeloid cells, thereby decreasing anti-tumor immunity compared to a subject without cancer or a subject with a cancer that does not interact with Tim4. In particular embodiments, interaction with Tim4 or Tim4 activity can be detected in the microenvironment of the tumor.

There is mounting evidence that myeloid cells at the tumor microenvironment (TME), including M2 macrophages, N2 neutrophils and myeloid-derived suppressor cells (MDSC), play a critical role in immune suppression by directly causing functional exhaustion of the cytotoxic T cells or by indirectly increasing the suppressive power of T-regulatory cells (Tregs). This leads to a skewed immunostimulatory versus immunosuppressive balance in the TME. The immunostimulatory environment of the TME is largely shaped by the presence of cytotoxic T cells and NK cells, cytolytic and phagocytosis-inducing M1 macrophages, cytotoxic N1 neutrophils, humoral response inducing B cells, and antigen presenting immunogenic dendritic cells (DC). Immunostimulatory cytokines and chemokines such as interferon gamma (IFN-γ), interleukin-12 (IL-12), tumor necrosis factor-alpha (TNF-α), etc. are key coordinators of the immunostimulatory activity. Important molecules that bias the immunosuppressive nature of the TME are anti-inflammatory Th2 cells, N2 neutrophils, M2 macrophages, Tregs, and tolerogenic DC. Immunosuppressive cytokines and chemokines such as transforming growth factor-beta (TGF-β), interleukin-10 (IL-10), macrophage colony stimulating factor (M-CSF), interleukin-4 (IL-4), etc. are key coordinators of the immunosuppressive activity. In specific embodiments, the Th1 response in a tumor microoenvironment is increased by administration of an anti-Tim4 antibody.

As described herein, the “tumor microenvironment” (TME) is the surrounding microenvironment that constantly interacts with tumor cells which is conducive to allow cross-talk between tumor cells and its environment. A tumor microenvironment plays a role in disrupting the cancer immunity cycle and plays a critical role in multiple aspects of cancer progression. For example, the TME can decrease drug penetration, confer proliferative and anti-apoptotic advantages to surviving cells, facilitate resistance without causing genetic mutations and epigenetic changes, and collectively modify disease modality and distort clinical indices. Without being limiting, the tumor microenvironment can include the cellular environment of the tumor, surrounding blood vessels, immune cells, fibroblasts, bone marrow derived inflammatory cells, lymphocytes, signaling molecules and the extracellular matrix. The tumor environment can include tumor cells or malignant cells that are aided and influenced by the tumor microenvironment to ensure growth and survival. The tumor microenvironment can also include tumor-infiltrating immune cells such as lymphoid and myeloid cells, which can stimulate or inhibit the antitumor immune response and stromal cells such as tumor-associated fibroblasts and endothelial cells that contribute to the tumor's structural integrity. Without being limiting, stromal cells can include cells that make up tumor-associated blood vessels, such as endothelial cells and pericytes, which are cells that contribute to structural integrity (fibroblasts), as well as tumor-associated macrophages (TAMs) and infiltrating immune cells including monocytes, neutrophils (PMN), dendritic cells (DCs), T and B cells, mast cells, and natural killer (NK) cells. The stromal cells make up the bulk of tumor cellularity while the dominating cell type in solid tumors is the macrophage.

Blocking of Tim4 or Tim4 peptides on the surface of myeloid cells can increase M1 macrophage polarization. For example, the M1 macrophage production can be increased by at least about 5%, 10%, 20%, 30%, 40%, 50%, 60%, 70%, 80%, 90%, 100%, 150%, 200% or greater when compared to the M1 macrophage level prior to administration of an anti-Tim4 antibody.

Macrophages may be classified by subsets: classically (M1) or alternatively (M2) activated macrophages (see, e.g., Laskin, Chem Res Toxicol. 2009 Aug. 17; 22(8): 1376-1385, the contents of which are hereby incorporated by reference in their entireties). Without wishing to be bound by theory, M1 macrophages are activated by standard mechanisms, such as IFNγ, LPS, and TNFα, while M2 macrophages are activated by alternative mechanisms, such as IL-4, IL-13, IL-10, and TGFβ. M1 macrophages can display a cytotoxic, proinflammatory phenotype, while M2 macrophages, suppress some aspects of immune and inflammatory responses and participate in wound repair and angiogenesis. In some embodiments, administration of an anti-Tim4 antibody can increase the level of M1 macrophages. In particular embodiments, administration of an anti-Tim4 antibody can increase the level of M1 macrophages in the tumor microenvironment.

In specific embodiments, the uptake of apoptotic cells is reduced following administration of an anti-Tim4 antibody. In specific embodiment, uptake of apoptotic cells is reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. Uptake of apoptotic cells can be determined by methods known in the art.

In certain embodiments, blocking of Tim4 can reduce pulmonary metastasis or micrometastases. For example, pulmonary metastasis or micrometastases can be reduced by about 5%, 10%, 20%, 30%, 40%, 50%, 60%, 70%, 80%, 90%, 100%, 150%, 200% or greater when compared to the pulmonary metastasis or micrometastases level prior to administration of an anti-Tim4 antibody.

The anti-Tim4 antibody as described herein can be administered according to methods described herein in a variety of means known to the art. The anti-Tim4 antibody can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body. Administration can be parenteral, pulmonary, oral, topical, intradermal, intramuscular, intraperitoneal, intravenous, subcutaneous, intranasal, epidural, ophthalmic, buccal, or rectal administration.

Any anti-Tim4 antibody as disclosed herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the anti-Tim4 antibody in a manner similar to that used for delivering insulin or chemotherapy to specific organs or tumors. Typically, using such a system, an anti-Tim4 antibody can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Anti-Tim4 antibodies can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006), Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

In specific embodiments, the anti-Tim4 antibody is formulated as a pharmaceutical composition. The pharmaceutical composition may be a liquid formulation or a solid formulation. When the pharmaceutical composition is a solid formulation it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip or a film. When the pharmaceutical composition is a liquid formulation it may be formulated as an oral solution, a suspension, an emulsion or syrup. Said composition may further comprise a carrier material independently selected from, but not limited to, the group consisting of lactic acid fermented foods, fermented dairy products, resistant starch, dietary fibers, carbohydrates, proteins, and glycosylated proteins. As used herein, the pharmaceutical composition could be formulated as a food composition, a dietary supplement, a functional food, a medical food, or a nutritional product as long as the required effect is achieved.

The pharmaceutical composition according to the invention, used according to the invention or produced according to the invention may also comprise other substances, such as an inert vehicle, or pharmaceutical acceptable adjuvants, carriers, preservatives etc., which are well known.

Generally, the dosage of the anti-Tim4 antibody will vary depending upon such factors as the patient's age, weight, height, sex, general medical condition and previous medical history. In specific embodiments, it may be desirable to administer the anti-Tim4 antibody in the range of from about 1 to 100 mg/kg, 20 to 30 mg/kg, 30 to 40 mg/kg, 40 to 50 mg/kg, 50 to 60 mg/kg, 60 to 70 mg/kg, 70 to 80 mg/kg, 80 to 100 mg/kg, 5 to 10 mg/kg, 2 to 10 mg/kg, 10 to 20 mg/kg, 5 to 15 mg/kg, 1 to 10 mg/kg, 1 to 5 mg/kg, 2 to 5 mg/kg or any range in between 1 and 100 mg/kg.

In some embodiments of the invention, the method comprises administration of multiple doses of the anti-Tim4 antibody. The method may comprise administration of 1, 2, 3, 4, 5, 6, 7, 8, 9, 10, 15, 20, 25, 30, 35, 40, or more therapeutically effective doses of the anti-Tim4 antibody. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to reduce the tumor size, tumor number, disease severity, or progression, and/or reduce uptake of apoptotic cells by myeloid cells. Changes in dosage may result and become apparent from the results of diagnostic assays for detecting tumor size, tumor number, disease severity, or progression, and/or reduce uptake of apoptotic cells by myeloid cells known in the art and described herein.

A “pharmaceutical composition” is a formulation containing the anti-Tim4 antibody in a form suitable for administration to a subject. In specific embodiments a pharmaceutical composition contains the anti-Tim4 antibody along with a non-natural carrier or excipient. In some embodiments, the pharmaceutical composition contains the anti-Tim4 antibody along with a natural carrier or excipient. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required. In specific embodiments the anti-Tim4 antibody is administered as a solution, dispersion, suspension, powder, capsule, tablet, pill, time release capsule, time release tablet, and/or time release pill.

Moreover, the administration may be by continuous infusion or by single or multiple boluses. In specific embodiments, an anti-Tim4 antibody can be infused over a period of less than about 4 hours, 3 hours, 2 hours or 1 hour. In still other embodiments, the infusion occurs slowly at first and then is increased over time.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

As used herein, “combination therapy” or “co-therapy” includes the administration of an anti-Tim4 antibody, as part of a specific treatment regimen intended to provide the beneficial effect from the co-action of the anti-Tim4 antibody with an additional composition. The beneficial effect of the combination includes, but is not limited to, pharmacokinetic or pharmacodynamic co-action resulting from the combination of these at least two compounds of the present invention. Administration of these at least two compounds of the present invention in combination typically is carried out over a defined time period (usually minutes, hours, days or weeks depending upon the combination selected).

“Combination therapy” also embraces the administration of the anti-Tim4 antibody as described herein in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the anti-Tim4 antibody and non-drug treatment is achieved. For example, in appropriate cases, the beneficial effect is still achieved when the non-drug treatment is temporally removed from the administration of the anti-Tim4 antibody, perhaps by days or even weeks.

In specific embodiments an anti-Tim4 antibody can be administered in combination with a chemotherapeutic agent. The chemotherapeutic agent (also referred to as an anti-neoplastic agent or anti-proliferative agent) can be an alkylating agent; an antibiotic; an anti-metabolite; a detoxifying agent; an interferon; a polyclonal or monoclonal antibody; an EGFR inhibitor; an FGFR inhibitor, a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase inhibitor), a cytidine analogue drug or any chemotherapeutic, anti-neoplastic or anti-proliferative agent.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar). In some embodiments, the additional chemotherapeutic agent can be a cytokine such as G-CSF (granulocyte colony stimulating factor).

In particular embodiments, an anti-Tim4 antibody as disclosed herein can be administered in combination with radiation therapy. Radiation therapy can also be administered in combination with an anti-Tim4 antibody and another chemotherapeutic agent described herein as part of a multiple agent therapy. In yet another aspect, an anti-Tim4 antibody may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar) or CMFP (cyclophosphamide, methotrexate, 5-fluorouracil and prednisone).

4. Methods of Producing Antibodies that Bind Tim4

Provided herein are methods for producing and isolating anti-Tim4 antibodies. In specific embodiments, the anti-Tim4 antibodies are monoclonal antibodies. In order to produce, isolate, or identify anti-Tim4 antibodies, a pool or library of test antibodies can be contacted with recombinant Tim4. The pool of antibodies can comprise at least 2 different kinds of monoclonal antibodies. In specific embodiments, the antibody pool comprises at least 3, at least 4, at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, or more test monoclonal antibodies. As used herein, a “test monoclonal antibody” or “test anti-Tim4 antibody” is any monoclonal antibody that is being tested for binding to recombinant Tim4. In certain embodiments, the recombinant Tim4 is recombinant murine Tim4 or recombinant human Tim4.

Test monoclonal antibodies can be contacted with recombinant Tim4 under standard conditions known in the art for antibody binding. Test monoclonal antibodies can be contacted with recombinant Tim4 for any amount of time necessary to determine binding. For example, test monoclonal antibodies can be contacted with recombinant Tim4 for about 1 min., 2 min., 3 min., 4 min., 5 min., 10 min., 15 min., 20 min., 25 min., 30 min., 35 min., 40 min., 45 min., 50 min., 55 min., 60 min., 1 hr., 1.5 hr., 2 hr., 3 hr., 4 hr., 5 hr., 6 hr., 7 hr., 8 hr., 9 hr., 10 hr., 11 hr., 12 hr., 15 hr., 18 hr., 24 hr., 36 hr., 48 hr., 30-60 min., 1-2 hr, 1-3 hr, 1-4 hr, 2-4 hr., or more. The temperature for determining binding can be any temperature sufficient to allow adequate binding of the test antibody to recombinant Tim4. For example, the temperature can be 25° C., 37° C., 55° C., or a range of 25-55° C., 25-37° C. or 37-55° C.

Following contacting, the binding solution containing unbound test monoclonal antibodies and any test antibodies bound to recombinant Tim4 can be washed to remove unbound test antibodies and any reagents necessary for the reaction. Test monoclonal antibodies that bind to recombinant Tim4 are selected as candidate monoclonal antibodies or candidate anti-Tim4 antibodies.

As used herein, “candidate monoclonal antibodies” refer to any test monoclonal antibody that binds recombinant human Tim4 or recombinant murine Tim4. In order to identify monoclonal anti-Tim4 antibodies that can block Tim4 on the surface of myeloid cells, candidate monoclonal antibodies that bind recombinant Tim4 can be contacted with myeloid cells in order to determine which candidate monoclonal antibodies inhibit the engulfment of dying cells by the myeloid cells. Antibodies that efficiently block Tim4 function can be identified by testing the blockage of the engulfment of apoptotic mouse thymocytes (treated with apoptosis inducer staurosporin) by human monocytic cell line (THP-1 cells). THP-1 cells pre-treated with candidate clones in serial dilution can be fed apoptotic thymocytes labeled with a membrane-bound fluorescent dye (PKH26) for an effective amount of time, such as 30 minutes. Dying cells that were not engulfed will be washed away, and THP-1 cells will be detached and analyzed by flow cytometry to determine the percentage of engulfment.

Candidate monoclonal antibodies can be contacted with recombinant Tim4 under standard conditions known in the art for antibody binding. Candidate monoclonal antibodies can be contacted with myeloid cells for any amount of time necessary to determine binding. For example, candidate monoclonal antibodies can be contacted with myeloid cells for about 1 min., 2 min., 3 min., 4 min., 5 min., 10 min., 15 min., 20 min., 25 min., 30 min., 35 min., 40 min., 45 min., 50 min., 55 min., 60 min., 1 hr., 1.5 hr., 2 hr., 3 hr., 4 hr., 5 hr., 6 hr., 7 hr., 8 hr., 9 hr., 10 hr., 11 hr., 12 hr., 15 hr., 18 hr., 24 hr., 36 hr., 48 hr., 30-60 min., 1-2 hr, 1-3 hr, 1-4 hr, 2-4 hr., or more. In specific embodiments, the myeloid cells used to select blocking candidate monoclonal antibodies are a macrophages or neutrophils. The temperature for determining blocking can be any temperature sufficient to allow adequate binding of the test antibody to recombinant Tim4. For example, the temperature can be 25° C., 37° C., 55° C., or a range of 25-55° C., 25-37° C. or 37-55° C.

Blocking can be determined by any method known in the art or disclosed elsewhere herein. Likewise, inhibition of dead or dying cells, such as apoptotic cells, can be determined by any method known in the art or disclosed elsewhere herein. The dead or dying cells measured in the method can be an apoptotic cell, such as an apoptotic thymocyte or a tumor-derived cell line disclosed elsewhere herein. In specific embodiments, candidate monoclonal antibodies are selected that inhibit engulfment of dead or dying cells, such as apoptotic cells. Candidate antibodies that inhibit engulfment of apoptotic cells by at least 5%, 10%, 20%, 30%, 40%, 50%, 60%, 70%, 80%, 90%, 95%, 100% or more can be identified as blocking antibodies. Further, candidate monoclonal antibodies that bind to recombinant Tim4 are selected as blocking monoclonal antibodies or blocking anti-Tim4 antibodies.

In specific embodiments, animals expressing recombinant Tim4 can be used to test the binding of candidate anti-Tim4 antibodies. In some embodiments, the animals expressing recombinant Tim4 do not express the endogenous Tim4 protein. For example, recombinant mice that do not express murine Tim4 can be engineered to express recombinant human Tim4 on the surface of myeloid cells. Any animal can be engineered to express human Tim4 without expressing the endogenous Tim4 protein. In specific embodiments, the animal is a laboratory animal such as a rodents, including mice or rats, pigs, cows, cats, dogs, non-human primates, or any other animal appropriate for producing myeloid cells that express recombinant Tim4. Macrophages from these animals can then be used to select candidate anti-Tim4 antibodies that inhibit the engulfment of apoptotic cells as described above.

As used herein, “blocking monoclonal antibodies” or “blocking anti-Tim4 antibodies” refer to any candidate monoclonal antibody that binds Tim4 on the surface of myeloid cells to inhibit the engulfment of dead or dying cells. Blocking antibodies can be co-cultured with dying cells (e.g., apoptotic cells) and myeloid cells in order to determine the inflammatory cytokine production by the myeloid cells. In specific embodiments, co-culturing the blocking antibodies with dying cells and myeloid cells can increase the inflammatory cytokine production by the myeloid cells compared to a proper control cell.

Accordingly, those antibodies that increase the inflammatory (i.e., pro-inflammatory) cytokine production by myeloid cells, after co-culture with of dead or dying cells, can be selected for administration to a subject. In specific embodiments, the antibodies selected by the method disclosed herein can be administered to a subject having a tumor. In some embodiments, monoclonal anti-Tim4 antibodies that reduce tumor growth in a subject are further selected. The subject can be a human subject or a laboratory animal. The laboratory animal can be a wild type animal or a transgenic animal. In specific embodiments, the monoclonal anti-Tim4 antibodies are tested for their ability to restrict tumor growth in an animal that expresses recombinant Tim4, but does not express the endogenous Tim4 protein. For example, monoclonal anti-Tim4 antibodies can be tested for their ability to restrict tumor growth in a mouse that does not express murine Tim4, but expresses human Tim4. A reduction in tumor growth, as used herein can be any reduction in tumor size, rate of tumor growth, or rate of tumor appearance. Such decreases or reductions in tumor growth can include, for example, at least a 5%, 10%, 20%, 30%, 40%, 50%, 60%, 70%, 80%, 90%, 95%, 100% or more. Selected antibodies that reduce tumor growth in a subject can be formulated as pharmaceutical compositions as disclosed elsewhere herein.

### Embodiments

1. A method of producing monoclonal antibodies that bind Tim4 on the surface of myeloid cells, said method comprising

a) contacting a pool of test monoclonal antibodies with recombinant murine Tim4 or recombinant human Tim4 and selecting candidate monoclonal antibodies that bind the recombinant murine Tim4 or recombinant human Tim4, respectively;

b) contacting the candidate monoclonal antibodies that bind the recombinant murine Tim4 or recombinant human Tim4 with myeloid cells and selecting blocking antibodies that inhibit the engulfment of dying cells; and

c) co-culturing the blocking antibodies that inhibit the engulfment of dying cells with dying cells and myeloid cells, measuring inflammatory cytokine production by the myeloid cells, and selecting blocking antibodies that increase inflammatory cytokine production by myeloid cells compared a proper control.

2. The method of embodiment 1, further comprising:

administering the selected blocking antibodies that increase inflammatory cytokine production to a mammal having a tumor and selecting those antibodies that reduces tumor growth in the mammal.

3. The method of embodiment 1, wherein the myeloid cells of step b) and/or the myeloid cells of step c) are macrophages.

4. The method of any one of embodiments 1-3, wherein the dying cells in step b) and/or step c) are apoptotic cells.

5. The method of embodiment 4, wherein the apoptotic cells are apoptotic thymocytes or a tumor-derived cell line.

6. The method of any one of embodiments 1-5, wherein the inflammatory cytokine is IL-1β, IL6, and/or TNFα.

7. The method of any one of embodiments 1-6, wherein the monoclonal antibodies bind recombinant human Tim4.

8. The method of any one of embodiments 1-6, wherein the monoclonal antibodies bind recombinant murine Tim4.

9. A pharmaceutical composition comprising an effective amount of an anti-Tim4 antibody, wherein the anti-Tim4 antibody is specific for Tim4 or peptides from Tim4.

10. The pharmaceutical composition of embodiment 9, wherein the anti-Tim4 antibody blocks Tim4 on the surface of at least one myeloid cell, following administration of the anti-Tim4 antibody to a subject.

11. The pharmaceutical composition of embodiment 9 or embodiment 10, wherein the anti-Tim4 antibody inhibits the engulfment of apoptotic cells by macrophages following administration of the anti-Tim4 antibody to a subject.

12. The pharmaceutical composition of embodiment 11, wherein the apoptotic cell is a thymocyte or a tumor-derived cell line.

13. The pharmaceutical composition of any one of embodiments 9-12, wherein the production of pro-inflammatory cytokines is increased following administration of the anti-Tim4 antibody to a subject.

14. The pharmaceutical composition of embodiment 13, wherein the inflammatory cytokines comprise at least one of IL-1β, IL6, and TNFα.

15. The pharmaceutical composition of any one of embodiments 9-14, wherein the anti-Tim4 antibody increases anti-tumor immunity in a subject having cancer, following administration of the anti-Tim4 antibody to the subject.

16. The pharmaceutical composition of any one of embodiments 9-15, wherein the anti-Tim4 antibody decreases cancer in a subject having cancer, following administration of the anti-Tim4 antibody to the subject.

17. An isolated nucleic acid molecule encoding the anti-Tim4 antibody of any one of embodiments 9-16.

18. A method of decreasing cancer in a subject having cancer, said method comprising:

administering to the subject an effective amount of a composition that is specific for Tim4 or peptides of Tim4, wherein the composition is an anti-Tim4 antibody.

19. The method of embodiment 18, wherein the anti-Tim4 antibody blocks Tim4 on the surface of at least one myeloid cell, following administration of the anti-Tim4 antibody to the subject.

20. The method of any one of embodiment 18-19, wherein the anti-Tim4 antibody inhibits the engulfment of apoptotic cells by macrophages following administration of the anti-Tim4 antibody to the subject.

21. The method of embodiment 20, wherein the apoptotic cell is a thymocyte or a tumor-derived cell line.

22. The method of any one of embodiments 18-21, wherein the production of pro-inflammatory cytokines is increased following administration of the anti-Tim4 antibody to the subject.

23. The method of embodiment 22, wherein the pro-inflammatory cytokines comprise at least one of IL-1β, IL6, and TNFα.

24. The method of any one of embodiments 18-23, wherein the anti-Tim4 antibody increases anti-tumor immunity in the subject, following administration of the anti-Tim4 antibody to the subject.

25. The method of any one of embodiments 10-16, wherein the decrease in cancer comprises a decrease in the size or number of tumors in the subject.

26. The method of any one of embodiments 18-25, wherein the decrease in cancer comprises a decrease in the rate of proliferation of cancer cells in the subject.

27. The method of any one of embodiments 18-26, wherein the cancer comprises B16F10 melanoma or Lewis Lung Carcinoma.

28. The method of any one of embodiments 18-27, further comprising administering to the subject an additional immunomodulatory treatment.

29. The method of embodiment 28, wherein the additional immunomodulatory treatment comprises administering to the subject a therapeutic vaccine, an immune checkpoint inhibitor, or an immune activator.

30. The method of any one of embodiments 18-29, further comprising administering to the subject a chemotherapy or radiation therapy.

31. A method of reducing the uptake of cells by myeloid cells, said method comprising:

contacting myeloid cells with a composition that is specific for Tim4 or a peptide of Tim4, wherein the composition is an anti-Tim4 antibody.

32. The method of embodiment 31, wherein the method reduces the uptake of apoptotic cells.

33. The method of embodiment 32, wherein the apoptotic cells are thymocytes or a tumor-derived cell line.

34. The method of any one of embodiments 31-33, wherein said contacting occurs in vitro and/or ex vivo.

35. The method of any one of embodiments 31-34, wherein the anti-Tim4 antibody blocks Tim4 on the surface of the myeloid cell.

36. The method of any one of embodiments 31-35, wherein the myeloid cell is a macrophage, dendritic cell, or a neutrophil.

37. The method of any one of embodiments 31-36, wherein the production of pro-inflammatory cytokines is increased following contacting of the anti-Tim4 antibody with the myeloid cells.

38. The method of embodiment 37, wherein the pro-inflammatory cytokines comprise at least one of IL-1β, IL6, and TNFα.

39. The method of embodiment 31, wherein said contacting comprises administering the anti-Tim4 antibody to a subject.

40. The method of embodiment 39, wherein anti-tumor immunity is increased in the subject, following administration of the anti-Tim4 antibody to the subject.

41. The method of any one of embodiments 39-40, wherein the subject has cancer and the cancer is decreased following administration of the anti-Tim4 antibody to the subject.

42. A method of increasing anti-tumor immunity in a subject having cancer, said method comprising:

administering to the subject an effective amount of a composition that binds Tim4, wherein the composition is an anti-Tim4 antibody.

43. The method of embodiment 42, wherein increasing anti-tumor immunity comprises at least one of: increased CD8+ T cells, increased CD4+ T cells, increased CD4+ or CD8+ T cells secreting interferon gamma, and decreased CD4+ T cells secreting IL10 among tumor infiltrating lymphocytes.

44. The method of embodiment 42 or 43, wherein the production of pro-inflammatory cytokines is increased following administration of the anti-Tim4 antibody to the subject.

45. The method of embodiment 43, wherein the pro-inflammatory cytokines comprise at least one of IL-1β, IL6, and TNFα.

46. Use of a composition that is specific for Tim4 or peptides of Tim4, in the treatment of cancer.

47. The use of embodiment 46, wherein said composition is an anti-Tim4 antibody.

48. Use of a composition that is specific for Tim4 or peptides of Tim4 in the manufacture of a medicament for the treatment of cancer.

49. The use of embodiment 48, wherein said composition is an anti-Tim4 antibody.

### EXPERIMENTAL

### Example 1. Tim-4 can Act as a Tolerogenic Signal that Suppresses Immune Response and Supports Tumor Growth

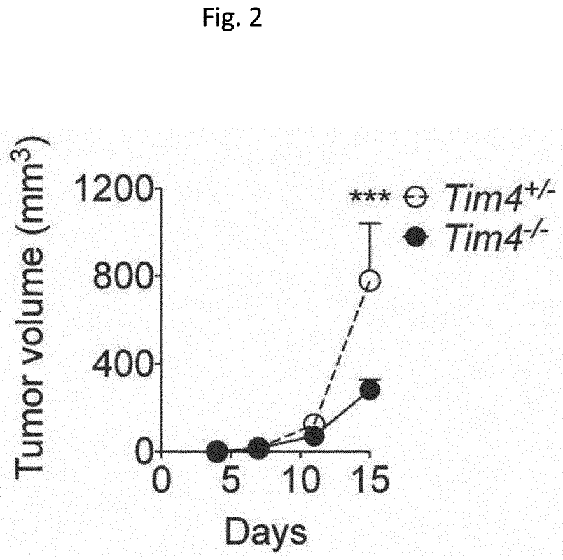

Efficient engulfment of dying cells by macrophages requires recognition of phosphatidylserine exposed in the surface of dying cells by TIM-4. To support a role for engulfment of dying tumor cells as a tolerogenic signal that suppresses immune responses and supports tumor growth, different tumor models were tested in Tim-4 deficient mice. Lewis Lung Carcinoma (LLC) cell line and B16F10 melanoma cell line were grown in complete DMEM media (10% fetal bovine serum (FBS), 200 mM L-glutamine and 100 units ml⁻¹ penicillin-streptomycin) at 37° C. with 5% CO₂. 1.0×10⁵ LLC cells were injected subcutaneously into the middle lower back of the mice. Tumor growth was measured using a digital caliper and tumor volume was estimated as length×width²×0.5 (FIG. 2).

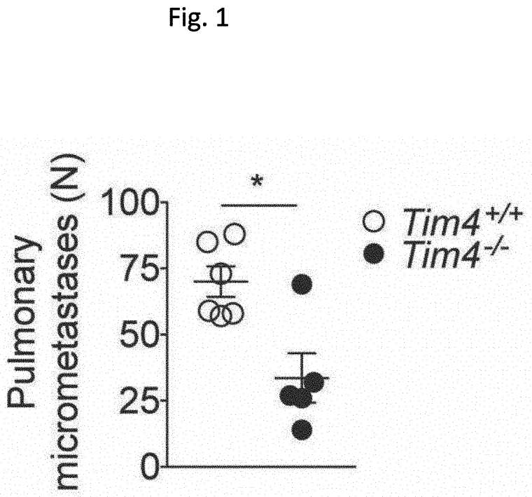

Alternatively, tumor metastasis was assessed by tail vein injection of 1.0×10⁵ B16F10 cells. Mice were euthanized 21 days post-injections, their lungs excised and all visible tumor spots onto the surface of all lobes were counted. We observed that the growth of both engrafted carcinoma cells and metastatic melanoma in the lungs was reduced in Tim4^{-/-} mice (FIG. 1 and FIG. 2). This further supports the conclusion that the engulfment of dying cells suppresses the inflammatory response to dying cells in the tumor.

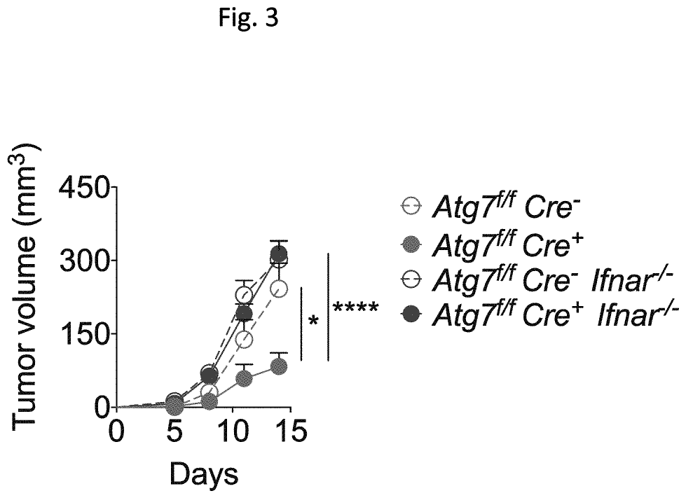

Induction of a type I IFN response in the tumor microenvironment can promote anti-tumor immunity. We therefore tested whether activation of type I IFN in responses in the absence of LAP participated in tumor suppression, by generating mice conditionally deficient in ATG7 and ubiquitously defective in Interferon Alpha/Beta Receptor 1 (Atg7f/f Ifnar−/−). In the absence of type I IFN signaling, tumor reduction in the absence of LAP was not observed (FIG. 3). Collectively, these findings are consistent with a role for TIM4-mediated engulfment of dying tumor cells and activation of LAP in triggering TAM suppressive function and tumor tolerance. In the absence of LAP, signaling in response to engulfed dying cells shifts to inflammation and activation of a type I IFN response, which is required for the elimination of tumor cells in this setting.

In order to quantitate metastatic spots, 1.0×10⁵ LLC cells, 2.5×10⁵ B16F10 cells and 5.0×10⁵ MC38 cells were injected subcutaneously into the middle lower back of the mice. Tumor growth was measured using a digital caliper and tumor volume was estimated as length×width²×0.5. Tumor metastasis was assessed by tail vein injection of 1.0×10⁵ B16F10 cells. Mice were euthanized 21 days post-injections, their lungs excised and all visible tumor spots onto the surface of all lobes were counted. (FIG. 1)

We claim:

1. A method of producing monoclonal antibodies that bind Tim4 on the surface of myeloid cells, said method comprising
a) contacting a pool of test monoclonal antibodies with recombinant murine Tim4 or recombinant human Tim4 and selecting candidate monoclonal antibodies that bind the recombinant murine Tim4 or recombinant human Tim4, respectively;
b) contacting the candidate monoclonal antibodies that bind the recombinant murine Tim4 or recombinant human Tim4 with myeloid cells and selecting blocking antibodies that inhibit the engulfment of dying cells; and
c) co-culturing the blocking antibodies that inhibit the engulfment of dying cells with dying cells and myeloid cells, measuring inflammatory cytokine production by the myeloid cells, and selecting blocking antibodies that increase inflammatory cytokine production by myeloid cells compared a proper control.

2. The method of claim 1, further comprising:
administering the selected blocking antibodies that increase inflammatory cytokine production to a mammal having a tumor and selecting those antibodies that reduces tumor growth in the mammal.

3. The method of claim 1, wherein the myeloid cells of step b) and/or the myeloid cells of step c) are macrophages.

4. The method of any one of claims 1-3, wherein the dying cells in step b) and/or step c) are apoptotic cells.

5. The method of claim 4, wherein the apoptotic cells are apoptotic thymocytes or a tumor-derived cell line.

6. The method of any one of claims 1-5, wherein the inflammatory cytokine is IL-1β, IL6, and/or TNFα.

7. The method of any one of claims 1-6, wherein the monoclonal antibodies bind recombinant human Tim4.

8. The method of any one of claims 1-6, wherein the monoclonal antibodies bind recombinant murine Tim4.

9. A pharmaceutical composition comprising an effective amount of an anti-Tim4 antibody, wherein the anti-Tim4 antibody is specific for Tim4 or peptides from Tim4.

10. The pharmaceutical composition of claim 9, wherein the anti-Tim4 antibody blocks Tim4 on the surface of at least one myeloid cell, following administration of the anti-Tim4 antibody to a subject.

11. The pharmaceutical composition of claim 9 or claim 10, wherein the anti-Tim4 antibody inhibits the engulfment of apoptotic cells by macrophages following administration of the anti-Tim4 antibody to a subject.

12. The pharmaceutical composition of claim 11, wherein the apoptotic cell is a thymocyte or a tumor-derived cell line.

13. The pharmaceutical composition of any one of claims 9-12, wherein the production of pro-inflammatory cytokines is increased following administration of the anti-Tim4 antibody to a subject.

14. The pharmaceutical composition of claim 13, wherein the inflammatory cytokines comprise at least one of IL-1β, IL6, and TNFα.

15. The pharmaceutical composition of any one of claims 9-14, wherein the anti-Tim4 antibody increases anti-tumor immunity in a subject having cancer, following administration of the anti-Tim4 antibody to the subject.

16. The pharmaceutical composition of any one of claims 9-15, wherein the anti-Tim4 antibody decreases cancer in a subject having cancer, following administration of the anti-Tim4 antibody to the subject.

17. An isolated nucleic acid molecule encoding the anti-Tim4 antibody of any one of claims 9-16.

18. A method of decreasing cancer in a subject having cancer, said method comprising:
administering to the subject an effective amount of a composition that is specific for Tim4 or peptides of Tim4, wherein the composition is an anti-Tim4 antibody.

19. The method of claim 18, wherein the anti-Tim4 antibody blocks Tim4 on the surface of at least one myeloid cell, following administration of the anti-Tim4 antibody to the subject.

20. The method of any one of claim 18-19, wherein the anti-Tim4 antibody inhibits the engulfment of apoptotic cells by macrophages following administration of the anti-Tim4 antibody to the subject.

21. The method of claim 20, wherein the apoptotic cell is a thymocyte or a tumor-derived cell line.

22. The method of any one of claim 18-21, wherein the production of pro-inflammatory cytokines is increased following administration of the anti-Tim4 antibody to the subject.

23. The method of claim 22, wherein the pro-inflammatory cytokines comprise at least one of IL-1β, IL6, and TNFα.

24. The method of any one of claims 18-23, wherein the anti-Tim4 antibody increases anti-tumor immunity in the subject, following administration of the anti-Tim4 antibody to the subject.

25. The method of any one of claims 10-16, wherein the decrease in cancer comprises a decrease in the size or number of tumors in the subject.

26. The method of any one of claims 18-25, wherein the decrease in cancer comprises a decrease in the rate of proliferation of cancer cells in the subject.

27. The method of any one of claims 18-26, wherein the cancer comprises B16F10 melanoma or Lewis Lung Carcinoma.

28. The method of any one of claims 18-27, further comprising administering to the subject an additional immunomodulatory treatment.

29. The method of claim 28, wherein the additional immunomodulatory treatment comprises administering to the subject a therapeutic vaccine, an immune checkpoint inhibitor, or an immune activator.

30. The method of any one of claims 18-29, further comprising administering to the subject a chemotherapy or radiation therapy.

31. A method of reducing the uptake of cells by myeloid cells, said method comprising:
contacting myeloid cells with a composition that is specific for Tim4 or a peptide of Tim4, wherein the composition is an anti-Tim4 antibody.

32. The method of claim 31, wherein the method reduces the uptake of apoptotic cells.

33. The method of claim 32, wherein the apoptotic cells are thymocytes or a tumor-derived cell line.

34. The method of any one of claims 31-33, wherein said contacting occurs in vitro and/or ex vivo.

35. The method of any one of claims 31-34, wherein the anti-Tim4 antibody blocks Tim4 on the surface of the myeloid cell.

36. The method of any one of claims 31-35, wherein the myeloid cell is a macrophage, dendritic cell, or a neutrophil.

37. The method of any one of claims 31-36, wherein the production of pro-inflammatory cytokines is increased following contacting of the anti-Tim4 antibody with the myeloid cells.

38. The method of claim 37, wherein the pro-inflammatory cytokines comprise at least one of IL-1β, IL6, and TNFα.

39. The method of claim 31, wherein said contacting comprises administering the anti-Tim4 antibody to a subject.

40. The method of claim 39, wherein anti-tumor immunity is increased in the subject, following administration of the anti-Tim4 antibody to the subject.

41. The method of any one of claims 39-40, wherein the subject has cancer and the cancer is decreased following administration of the anti-Tim4 antibody to the subject.

42. A method of increasing anti-tumor immunity in a subject having cancer, said method comprising:
administering to the subject an effective amount of a composition that binds Tim4, wherein the composition is an anti-Tim4 antibody.

43. The method of claim 42, wherein increasing anti-tumor immunity comprises at least one of: increased CD8+ T cells, increased CD4+ T cells, increased CD4+ or CD8+ T cells secreting interferon gamma, and decreased CD4+ T cells secreting IL10 among tumor infiltrating lymphocytes.

44. The method of claim 42 or 43, wherein the production of pro-inflammatory cytokines is increased following administration of the anti-Tim4 antibody to the subject.

45. The method of claim 43, wherein the pro-inflammatory cytokines comprise at least one of IL-1β, IL6, and TNFα.

46. The use of a composition that is specific for Tim4 or peptides of Tim4, in the treatment of cancer.

47. The use of claim 46, wherein said composition is an anti-Tim4 antibody.

48. The use of a composition that is specific for Tim4 or peptides of Tim4 in the manufacture of a medicament for the treatment of cancer.

49. The use of claim 48, wherein said composition is an anti-Tim4 antibody.


SEQUENCE LISTING (4 sequences)

SEQ ID NO: 1 - DNA, 1306 bp, Homo sapiens
  misc_feature (1)..(1306): Human Tim-4 nucleic acid sequence (NCBI Accession BC008988)
ataagaggttgggctttggatagatagacagactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgctgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttaaaaaaaaaaaaaaa

SEQ ID NO: 2 - protein, 378 aa, Homo sapiens
  MISC_FEATURE (1)..(378): Human Tim-4 amino acid sequence (NP_612388.2)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 3 - DNA, 1032 bp, Mus musculus
  misc_feature (1)..(1032): Mouse Tim-4 nucleic acid CDS sequence (ACCESSION   NM_178759 mRNA)
atgtccaaggggcttctcctcctctggctggtgacggagctctggtggctttatctgacaccagctgcctcagaggatacaataatagggtttttgggccagccggtgactttgccttgtcattacctctcgtggtcccagagccgcaacagtatgtgctggggcaaaggttcatgtcccaattccaagtgcaatgcagagcttctccgtacagatggaacaagaatcatctccaggaagtcaacaaaatatacacttttggggaaggtccagtttggtgaagtgtccttgaccatctcaaacaccaatcgaggtgacagtggggtgtactgctgccgtatagaggtgcctggctggttcaatgatgtcaagaagaatgtgcgcttggagctgaggagagccacaacaaccaaaaaaccaacaacaaccacccggccaaccaccaccccttatgtgaccaccaccaccccagagctgcttccaacaacagtcatgaccacatctgttctcccaaccaccacaccaccccagacactagccaccactgccttcagtacagcagtgaccacgtgcccctcaacaacacctggctccttctcacaagaaaccacaaaagggtccgccttcactacagaatcagaaactctgcctgcatccaatcactctcaaagaagcatgatgaccatatctacagacatagccgtactcaggcccacaggctctaaccctgggattctcccatccacttcacagctgacgacacagaaaacaacattaacaacaagtgagtctttgcagaagacaactaaatcacatcagatcaacagcagacagaccatcttgatcattgcctgctgtgtgggatttgtgctaatggtgttattgtttctggcgtttctccttcgagggaaagtcacaggagccaactgtttgcagagacacaagaggccagacaacactgaagatagtgacagcgtcctcaatgacatgtcacacgggagggatgatgaagacgggatcttcactctctga

SEQ ID NO: 4 - protein, 343 aa, Mus musculus
  MISC_FEATURE (1)..(343): Mouse Tim-4 amino acid sequence (NP_848874.3)
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLRGKVTGANCLQRHKRPDNTEDSDSVLNDMSHGRDDEDGIFTL